(19) United States
(12) Patent Application Publication
(10) Pub. No.: US 2007/0111328 A1
(43) Pub. Date: May 17, 2007

(54) Scintillation proximity assay for the identification of P-glycoprotein modulators

(75) Inventors: Laishun Chen, Princeton, NJ (US); Gang Luo, Plainsboro, NJ (US); Saeho Chong, Princeton, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 11/506,667

(22) Filed: Aug. 18, 2006

Related U.S. Application Data
(60) Provisional application No. 60/710,010, filed on Aug. 19, 2005.

(51) Int. Cl. G01N 33/543

(52) U.S. Cl. 436/518

(57) ABSTRACT

The present invention relates to methods for the identification and characterization of compounds that modulate P-glycoprotein (P-gp) and the use of such methods for the identification and characterization of therapeutic agents. The invention further relates to a kit for performing such methods.

This application claims priority to U.S. Provisional Patent Application Ser. No. 60/710,010, filed Aug. 19, 2005, which is hereby incorporated by reference in its entirety.

### FIELD OF THE INVENTION

The subject matter disclosed and claimed herein relates to methods for the identification and characterization of compounds that modulate P-glycoprotein (P-gp) and the use of such methods for the identification and characterization of therapeutic agents.

### BACKGROUND OF THE INVENTION

P-Glycoprotein (P-gp) is the most extensively studied ATP-binding cassette (ABC) transporter and comprises two homologous and symmetrical halves, each of which contains six transmembrane domains that are separated by an intracellular flexible linker polypeptide loop with an ATP-binding motif (Gottesman, M. M. and I. Pastan, Annu. Rev. Biochem., 1993. 62:385-427.). P-gp is expressed on the canalicular surface of hepatocytes in liver, the apical surface of epithelial cells of intestine and placenta, the apical surface of epithelial cells of proximal tubules in kidneys, and the luminal surface of capillary endothelial cells in brain.

Essentially, P-gp functions as a biological barrier by extruding toxins and xenobiotics out of cells and organs, by excreting these toxins into bile, intestinal lumen, and urine, and by preventing toxin accumulation in brain. Many experimental studies show that P-gp plays a significant role in the processes of drug absorption, distribution, excretion, and even metabolism in humans and other species (Borst, P., et al., Pharmacol. Ther., 1993. 60(2):289-99; Borst, P. and A. H. Schinkel, Eur. J. Cancer, 1996. 32A(6):985-90; Lum, B. L. and M. P. Gosland, Hematol. Oncol. Clin. North Am., 1995. 9(2):319-36; Troutman, M. D., et al., Drug-Drug Interactions, ed. A. D. Rodrigues. 2001, New York: Marcel Dekker, Inc. 295-357). Furthermore, inhibition, as well as induction, of P-gp is the root cause of drug-drug interactions, which could lead to significant alterations in the pharmacokinetic profiles of these drugs. Therefore, identification of P-gp substrates and inhibitors becomes necessary for prediction of possible P-gp mediated drug-drug interactions in clinics.

There are many screening assays, including cell-based (Caco-2 cells, Madine-Darby canine kidney cells and brain microvessel endothelial cells), substrate transport (Hochman, J. H., et al., Curr. Drug Metab., 2002. 3(3):257-73), competitive inhibition (Troutman, M. D., et al., 2001; Cavet, M. E., et al., Br. J. Pharmacol., 1996. 118(6):1389-96), membrane vesicle uptake (Kamimoto, Y., et al., J. Biol. Chem., 1989. 264(20):11693-8), photolabeling (Greenberger, L. M., J. Biol. Chem., 1993. 268(15):11417-25; Borchers, C., et al., Mol. Pharmacol., 2002. 61(6):1366-76), ATPase (Garrigues, A., et al., Anal. Biochem., 2002. 305(1):106-14; Adachi, Y., et al., Pharm. Res., 2001. 18(12):1660-8), and radioligand binding (Doppenschmitt, S., et al., J. Pharmacol. Exp. Ther., 1999. 288(1):348-57; Martin, C., et al., Biochemistry, 2000. 39(39):11901-6; Martin, C., et al., Biochemistry, 2001. 40(51):15733-42).

However, none of the aforementioned assays is able to serve for high throughput screening. The present invention provides a method for identifying and characterizing P-gp substrates and/or inhibitors with high throughput screening capability.

### SUMMARY OF THE INVENTION

The subject matter disclosed and claimed herein relates to methods for the identification and characterization of compounds that modulate P-glycoprotein (P-gp) and the use of such methods for the identification and characterization of therapeutic agents. Such methods provide screening methods for identification of P-gp substrates and/or inhibitors with high throughput capacity, adequate sensitivity, and cost effectiveness.

Briefly, P-gp (prepared from KB-V1 cells which highly expresses P-gp in the presence of vinblastine) was incubated with WGA-Ysi scintillation proximity assay (SPA) beads pre-coated with scintillant for one hour in a 96-well plate. The test compound and radiolabeled probe [³H]vinblastine was then added to the incubation mixture. The samples were incubated for an additional 3 to 24-hours. The binding of [³H]vinblastine to P-gp was measured using a Perkin-Elmer MicroBeta counter. P-gp substrates or inhibitors decreased the binding signal by competing with [³H]vinblastine at the binding site or inhibiting the binding of [³H]vinblastine to P-gp.

One embodiment disclosed herein is a method for identifying a test compound capable of modulating the activity of P-glycoprotein, comprising: (a) incubating P-glycoprotein (P-gp) and scintillation proximity assay (SPA) beads under conditions which permit the formation of a complex between said P-gp and said SPA beads; (b) introducing a test compound to the complex; (c) introducing a radiolabeled probe to the complex; and (d) measuring the energy produced by the interaction of the radiolabeled probe with the SPA beads to determine if the test compound is a modulator of P-gp. An additional embodiment further comprises the step of measuring the energy produced by the interaction of the radiolabeled probe with the SPA beads prior to the introduction of a test compound. The test compound may be identified as a modulator of P-gp when the amount of energy produced in the absence of the test compound is different than the amount of energy produced in the presence of the compound. Further, the energy, which may be light, may be produced by a scintillant coated onto the SPA beads. The energy may be measured by fluorescent, luminescent, radioactive, or absorbance readout.

The radiolabeled probe may be [³H]-vinblastine (or a vinblastine analog), and the energy may be produced at a [³H]-vinblastine/analog concentration of between about 10 nM and about 1000 nM and may be produced at a [³H]-vinblastine/analog concentration of about 30 nM. The P-gp may be prepared from a human cell line, such as KB-V1. Further, the SPA beads may be yttrium silicate-wheat germ agglutinin (Ysi-WGA) beads and may be present in an amount of between about 50 μM to about 2000 μg, and may be present in an amount of about 400 μg. The complex may be formed by incubating the P-gp and the SPA beads in the presence of about 400 μM of phenylmethylsulphonyl fluoride in about 50 mM Tris-HCl buffer, and may be formed at a temperature of between about 4° C. and about 45° C. The complex may be formed at a temperature of about 20° C. and incubation may occur for between about 0.01 hours and about 6 hours and for about 1 hour. The test compound may be a substrate and/or an inhibitor of P-gp.

Another embodiment is a method for identifying a test compound capable of modulating the activity of P-glycoprotein, comprising: (a) incubating P-glycoprotein (P-gp) and scintillation proximity assay (SPA) beads coated with a scintillant under conditions which permit the formation of a complex between the P-gp and the SPA beads; (b) introducing a test compound to the complex; (c) introducing [³H]-vinblastine or analog thereof; and (d) measuring the light produced by the interaction of the [³H]-vinblastine/analog with the SPA beads to determine if the test compound is a modulator of P-gp.

Another embodiment is a method for identifying a test compound capable of modulating the activity of P-glycoprotein, comprising: (a) incubating P-glycoprotein (P-gp) and scintillation proximity assay (SPA) beads coated with a scintillant under conditions which permit the formation of a complex between the P-gp and the SPA beads, wherein the SPA beads are yttrium silicate-wheat germ agglutinin (Ysi-WGA) beads; (b) introducing a test compound to the complex; (c) introducing [³H]-vinblastine or an analog thereof; and (d) measuring the light produced by the interaction of the [³H]-vinblastine/analog with the SPA beads to determine if the test compound is a modulator of P-gp.

Another embodiment is a kit for performing any of the above methods. For example, an embodiment is a for identifying a test compound capable of modulating the activity of P-glycoprotein, comprising: (a) P-glycoprotein; (b) scintillation proximity assay beads, and (c) radiolabeled probe. The radiolabeled probe may be [³H]-vinblastine or an analog thereof, and the SPA beads may be yttrium silicate-wheat germ agglutinin (Ysi-WGA) beads. Another example is a kit for identifying a test compound capable of modulating the activity of P-glycoprotein, comprising: (a) P-glycoprotein; (b) yttrium silicate-wheat germ agglutinin beads, and (c) [³H]-vinblastine or analog thereof.

### BRIEF DESCRIPTION OF THE DRAWINGS

FIGS. 1A-C show P-gp expression in KB-V1cells and the parental cell line, KB 3-1 cells, examined by FACS.

FIG. 2 shows P-gp expression in the membranes prepared from KB-V1 and KB-3-1 cells with Western blot analysis.

FIG. 3 shows the effect of SPA beads on background signals obtained from PVT-WGA and YSI-WGA beads in the presence of 0.1 μCi or 0.5 μCi [³H]vinblastine.

FIG. 4 shows the ratio of signal to noise obtained with various P-gp radiolabeled probes.

FIG. 5 shows the ratio of signal to noise obtained for WGA-Ysi beads only, WGA-Ysi beads and KV-3-1, and WGA-Ysi beads and KB-V1 membrane preparations in the presence of radiolabeled probe [³H]vinblastine only and [³H]vinblastine plus non-radiolabeled vinblastine (100 μM).

FIG. 6 shows the effect of radioligand amount on total and non-specific binding signals.

FIG. 7 shows the effect of membrane sources and amount on the signals. With KB-3-1 cell membranes, both specific and non-specific signals were not altered by various amount of membrane proteins (A); in the presence of KB-V1 cell membrane, the specific signal reached to a peak value at 8 μg protein whereas non-specific signal increased insignificantly (B). VB, vinblastin; 3-1 MEM, KB-3-1 cell membrane; V1 MEM, KB-V1 cell membrane.

FIG. 8 shows the signal obtained with test compounds. The final concentration of the test compounds was 10 μM. Other conditions are described in Example 3. Data represents the mean of triplicates.

FIG. 9 shows the inhibition curves obtained with selected P-gp substrates/inhibitors. Experimental conditions were as described in Example 3. All the data points represent the mean of triplicates. The curves were fitted using the PRISM Software and IC50 values were calculated.

FIG. 10 shows the variability of specific and non-specific binding signals. Experimental conditions are as described in the Example 3.

### DETAILED DESCRIPTION OF THE INVENTION

The subject matter disclosed and claimed herein relates to methods for the identification and characterization of compounds that modulate P-glycoprotein (P-gp) and the use of such methods for the identification and characterization of therapeutic agents. Further provided is a screening method for identification of P-gp substrates and/or inhibitors with high throughput capacity, adequate sensitivity, and cost effectiveness.

The methods described herein are based on the scintillation proximity assay (SPA) technique and its use to detect signal changes in the presence of a test compound. The signal obtained in the absence of a test compound represents the binding of radiolabeled probe [³H]vinblastine, or an analog thereof, to P-gp and the signal obtained in the presence of a test compound represents the effect of a test compound on the binding. The signal should be reduced when the test compound is a P-gp substrate and/or inhibitor because of competition on the binding site or inhibition of the binding.

SPA is a homogenous assay by which the binding signal could accurately be detected without separation of bound and free radiolabeled ligand. Lack of a separation step makes it possible to automatically perform the assay in high throughput format for screening many samples. In addition, the lack of a separation step also avoids the shift of the apparent equilibration of the binding event caused by filtration during the wash step.

P-gp can be obtained from cell membranes of many cell lines. However, it is important to prepare cell membranes from cells which highly express P-gp to achieve acceptable signal to noise ratio. Additionally, use of human cells may be necessary because the lipid components of the membranes are known to affect the binding affinity and the lipid components of non-human cell types may be different and potentially impact binding kinetics. Several sources of cells were evaluated and it was found that cell membranes prepared from KB-V1 cells gave a satisfactory signal to noise ratio.

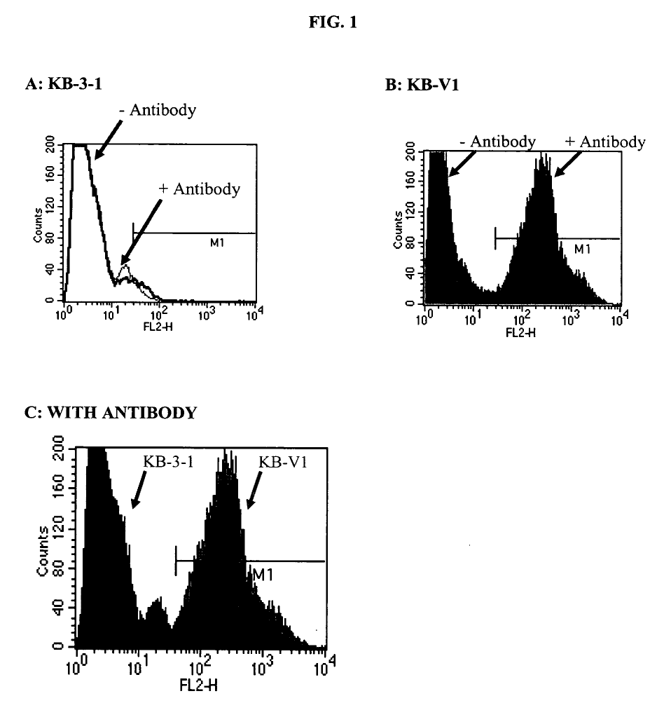

The KB-V1 cell line highly expresses P-gp and is a multidrug-resistant subclone derived from KB-3-1 cells, which were derived from HELA cells, which in turn were derived from a human cervix carcinoma. A fluorescence-activated cell sorter (FACS) assay was used to evaluate the expression level of P-gp (FIG. 1). As shown in FIG. 1A, Parental KB-3-1 cells displayed no difference in the fluorescence density in the presence and absence of a specific antibody against human P-gp (MRK-16) suggesting negligible expression of P-gp in KB-3-1 cells. However, treatment of the KB-V1 cells with the antibody showed more than 100-fold fluorescent signal shift in comparison to buffer treatment (FIG. 1B). FIG. 1C presents the comparison between KB-3-1 and KB-V1 cells in the presence of MRK-16. These results consistently indicate the strong expression of P-gp in the KB-V1 cells. All the results were obtained using membrane protein prepared from KB-V1 and KB-3-1 cells.

Cell culture conditions for the two cell lines were adapted from the supplier's instructions, and are described in Example 1. In order to sustain high expression of P-gp in the KB-V1 cells, it is necessary to maintain vinblastine concentration (100 to 1000 μg/ml) in the culture media. In preferred embodiments, the vinblastine concentration was 1000 μg/ml.

High background signal caused by non-specific binding is a common obstacle for developing an assay based on molecular binding principles, especially for compounds having lipophilic properties such as P-gp substrates. Non-specific binding needs to be addressed when employing SPA-based technology. In order to decrease the non-specific signal, crude membranes prepared from the disruption of KB-V1 cells with hypotonic force were further purified by sucrose gradient centrifugation. This procedure should remove irrelevant lipids or proteins contained in the cell fractions and, therefore, increase P-gp density/unit of total protein in the final membrane preparation. Consequently, this would maximize the signal representing the interaction between P-gp and radiolabeled ligand [³H] vinblastine, and increase the signal to noise ratio.

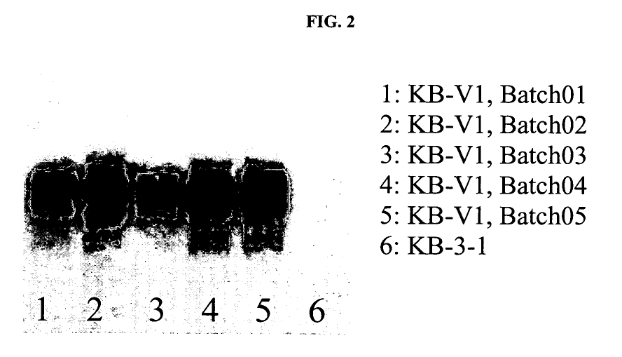

P-gp density in the resultant membranes of several batches was assessed with Western blot analysis using the human P-gp specific antibody C219. FIG. 2 shows P-gp expression in the membranes prepared from KB-V1 and B-3-1 cells with Western blot analysis. Five batches of KB-V1 cell membranes and one batch of KB-3-1 cell membranes were examined. As shown in FIG. 2, all the batches of cell membranes prepared from KB-V1 cells had much higher density of P-gp compared to KB-3-1 cells. The preparation protocol is described in the Example 2.

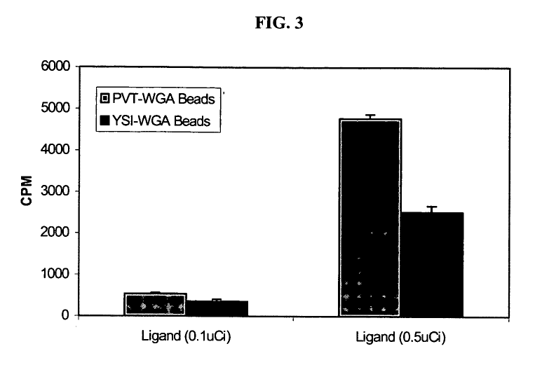

FIG. 3 represents the comparison of the results obtained from yttrium silicate-wheat germ agglutinin (Ysi-WGA; Amersham Inc.) and PVT-WGA beads. When-radioligand (0.1 μCi or 0.5 μCi) was added into the reaction mixture, the background signal was lower from Ysi-WGA than from PVT-WGA. The specific binding signal reached plateaus at 3-4 hours after the addition of radioligand, and was stable up to 24 hours. An experiment was performed to optimize the amount of Ysi-WGA beads and 400 μg beads/well was found to be optimal.

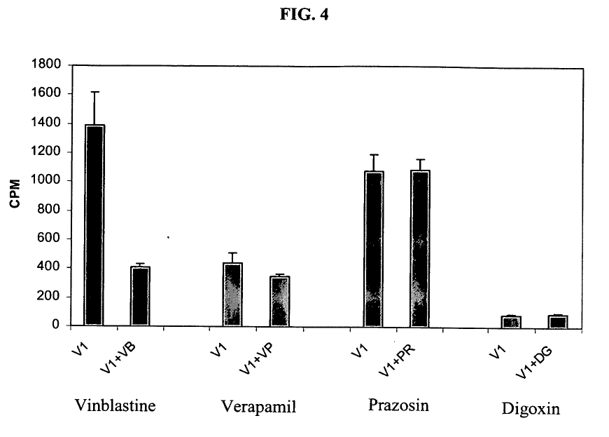

Use of a radioligand having a high specific activity and binding affinity for P-gp was an experimental parameter considered when establishing the experimental conditions for the methods disclosed and claimed herein. Several radiolabeled P-gp probes were tested including [³H]-vinblastine, [³H]-verapamil, [³H]-prazosin, and [³H]-digoxin. Among them, only [³H]-vinblastine purchased from Amersham Inc gave a satisfactory signal to noise ratio. (FIG. 4).

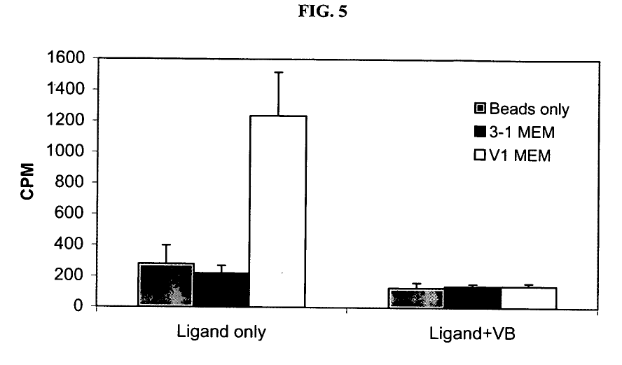

As shown in FIG. 5, the total signal generated from Ysi-WGA beads and KB-V1 cell membranes in the presence of radiolabeled ligand [³H]-vinblastine only was approximately six fold of the background signal obtained in presence of both radioligand [³H]-vinblastine and 100 μM of non-radiolabeled vinblastine, whereas total signal generated from beads alone, or beads and control membranes (KB-3-1 cell membranes) in the presence of radiolabeled ligand [³H]-vinblastine only, was close to that obtained in the presence of both radioligand [³H]-vinblastine and 100 μM of non-radiolabeled vinblastine. The results provided additional evidence that the signal was attributed to the specific binding between the probe and P-gp. Therefore, in the present invention, [³H]-vinblastine was chosen as radiolabeled ligand.

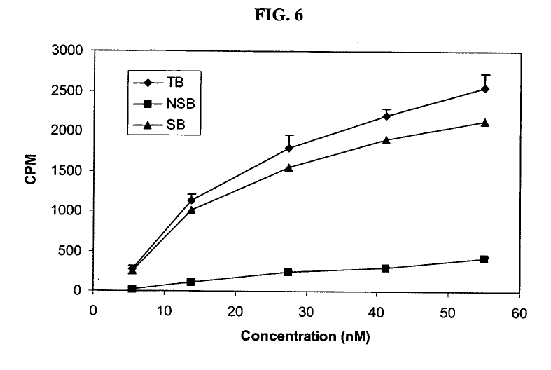

The binding curve of [³H]-vinblastine at the concentrations of 5 to 55 nM to P-gp is shown in FIG. 6, where a typical ‘saturation isotherm’ for total binding (TB) and specific binding (SB) was demonstrated. The specific signal values versus the amount of radioligand was fitted to the equation using the Prism software (GraphPad Software Inc, San Diego, Calif.) as the following:

$$B = {B}_{max} \cdot \frac{[ L ]}{{K}_{D} + [ L ]}$$

where Bₘₐₓ is the maximal binding, [L] is a ligand concentration, and K_{D} is the binding dissociation constant. The fitting gave the K_{D} value of 4 nM, which is in agreement with literature data. Upon further optimization, 0.04 μCi (equivalent to 10-30 nM, dependent on specific radioactivity of vinblastine received from the manufacture) was selected to give satisfactory readouts with respect to the signal to noise ratio and the signal window.

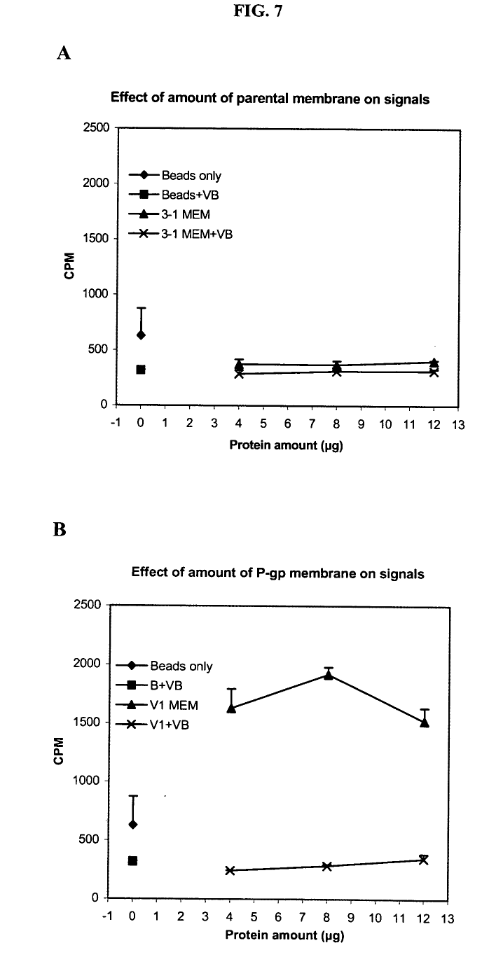

An experiment was performed to optimize cell membrane amounts used in the reactions. As shown in FIG. 7, for control membranes at the range of 4-12 μg/well, the total signals obtained in the presence of [³H]-vinblastine only were close to the background signals obtained in the presence of both [³H]-vinblastine and 100 μM non-radiolabeled vinblastine. However, for KB-V1 cell membranes at the range of 4-12 μg/well, the total signals obtained in the presence of [³H]-vinblastine only were much higher than background signals obtained in the presence of both [³H]-vinblastine and 100 μM non-radiolabeled vinblastine. With consideration of both cell membrane amounts and signal window, 6 μg of cell membrane proteins was chosen as the optimal amount for the invention. However, this optimized amount may vary from batch to batch due to the variation of P-gp expression level in the cells.

The sequence of addition of reaction components should be considered to minimize background noise in the assay. Pre-incubation of P-gp membranes with beads reduced non-specific signal generated from the binding of the labeled probe to the beads. However, the extent of reduction by the pre-incubation procedure is dependent on the radioligand purity which can vary from batch to batch. Certain batches have more impurities causing increased non-specific binding. It is believed that the pre-incubation could minimize interaction of radioligand with the naked beads as a result of the membranes enveloping the beads. Pre-incubating the membranes and beads consistently produced satisfactory signal/noise ratio. The assay procedure is described in Example 3.

### EXAMPLES

### Example 1

### Culture Media and Reagent Preparation

The media used in the methods disclosed and claimed herein include: Dulbecco's Modified Eagle Medium (DMEM; Cat # 11995-065, 500 ml) containing 15% (v/v) Fetal Bovine Serum (FBS, Gibco, Cat # 26140-087, 100 ml)m used for KB-V1 cells; and DMEM containing 10% (v/v) FBS which is used for KB-3-1 cells.

Additionally, solutions of vinblastine were prepared. To do so, vinblastine (Vinblastine sulfate salt, Sigma, Cat # V-1377, 25 mg) solution (1 mg/ml) was made in distilled H₂O. Filter the vinblastine solution using a 0.22 μm filter. Store at 20° C., and thaw at 37° C. just prior to use.

The cell culture procedure includes steps a)-d). Step a): obtain KB-V-1 and KB-3-1 cells (in 2 ml tubes) from frozen storage and quickly thaw to 37° C. Step b): transfer the cells to a 25 cm² T-flask with vented cap (Becton Dickson, Cat # 3808 or 381353136, one tube per one 25 cm² T-flask) containing 5 ml of either cell culture medium (pre-warmed to 37° C. for 30 minutes). It is preferable not to treat the KB-V-1 cells with vinblastine for the first culture passage since the cells may become unstable. Note that KB-3-1 cells grow faster than KB-V-1 cells and the KB-3-1 cells may reach 90% confluency (observed under the light microscope) in 3 or 4 days, whereas it may take one week for KB-V-1 cells to reach 90% confluency. Step c): once the cells reach no more than 90% confluency, split the cells. The cells should not reach more than 90% confluency.

The procedure used to split the cells includes: aspirating the culture medium; washing the cells with 5 ml of sterile phosphate buffered saline (PBS, Cellgro, Cat # 21-040-CV, 500 ml); adding 3 ml Trypsin-EDTA (GibcoBRL, Cat # 25200-056, 100 ml); and placing the T-flask in 37° C. for 2-3 minutes for digestion. Following digestion, remove the T-flask and add 10 ml fresh culture medium (warmed) into the T-flask in the hood. Transfer the cells into a 50 ml centrifuge tube (Corning, Cat # 25330-50) and centrifuge at 1000 rpm for 2 minutes (Eppendorf Centrifuge, 5804). Following centrifugation, aspirate the supernatant, add 5 ml of fresh cell culture medium, and mix the cells by pipetting the medium about 10-15 times. Following mixing of the cells, set up the cell culture in 175 cm² T-flask (BD, Cat # 353112) at the ratio of 5 to 10. The total medium volume could be 50-60 ml.

Following setting up the culture flasks, add the vinblastine solution (as described above) to the T-flask at a final concentration of 1 μl/ml. Again, KB-3-1 cells grow faster than KB-V-1 cells and this is especially true when vinblastine is used (the second passage). Significant cell death will likely occur during the second passage. KB-V-1 cells will grow fast in the subsequent cell passages. Note that since the cells are sensitive to pH fluctuation, the culture medium should be changed regularly. It is suggested that the cell culture medium be replaced every three to four days for KB-3-1 cells and every four to five days for KB-V-1 cells. Vinblastine could be added into the T-flask individually or into the medium container (bottle).

Cells should be harvested for membrane preparation when they reach more than 90% confluency. It is expected that the yield of cells will be high, though care should be taken when handling the cells to avoid inducing cell death. To harvest the cells, aspirate the medium, and wash the cells with PBS (pH 7.4, Sigma, Cat # P-3813) twice. Scrape the cells with a long arm scraper (Becton Dickson, Cat # 353087) and add 5 ml PBS. Transfer the cells into 50 ml centrifuge tube (Corning, Cat # 25330-50). Rinse the T-flask with 5 ml of PBS once more and transfer to the 50 ml centrifuge tube.

Cells collected will be used for Membrane Preparation (Example 2).

### Example 2

### Membrane Preparation

Following harvest of the cell cultures as described in Example 1, discard the culture medium, and twice-rinse the flask with 5-10 ml of PBS-PMSF (containing 400 μM of PMSF). Scrape cells with a long-arm scraper in the culture flask and add 5 ml PBS-PMSF and then transfer the cell suspension into a 50 ml centrifuge tube on ice. Rinse the flask with another 5 ml PBS-PMSF and transfer to the centrifuge tube.

Once the cells are collected, spin the cells in the centrifuge the tube at 1200 g for 5 minutes and aspirate the supernatant. Resuspend the cells in 40 ml hypotonic lysis buffer (1 mM NaHCO3, pH 7.0, containing 200 μM PMSF, 5 μg/ml leupeptin, 5 μg/ml aprotinin, and 1 μg/ml pepstatin A) and gently stir the mixture at 4° C. for 1 hour. Following the one hour incubation, centrifuge the suspension at 100,000 g (35,000 rpm; 50.2 Ti rotor; Beckman L-70 ultracentrifuge) for 30 minutes at 4° C. Following this centrifugation step, add 5 ml hypotonic lysis buffer to the tube, and transfer the pellet to a 15 ml glass-glass tight homogenizer. Wash the remainder of any cells from the tube with another 5 ml hypotonic lysis buffer and transfer the wash to the homogenizer. The total volume in the homogenizer should be about 15 ml. Stroke the cell suspension 30 times (3 times/minute) on ice and dilute the resultant suspension with 1 volume of homogenate buffer (Sucrose 520 mM, CaCl2 0.4 mM, HEPS 10 mM, Tris-Base 10 mM, pH 7.4).

Following homogenization, centrifuge the mixture at 1200 g for 8 minutes at 4° C., and discard the pellet. Dilute the supernatant 2 fold with suspension buffer (Sucrose 260 mM, CaCl₂ 0.2 mM, HEPS 5 mM, and Tris-Base 5 mM, pH 7.4) and centrifuge at 100,000 g (35,000 rpm with a 50.2 Ti rotor at Beckman L-70 ultracentrifuge) for 45 minutes at 4° C. Following centrifugation, homogenize the pellet with 15 ml of suspension buffer in a glass-glass tight Dounce homogenizer on ice (50 stroke) and add 3 ml of resulting suspension to gradient centrifuge tubes. Gently add 9 ml of 38% sucrose from the bottom using a long syringe needle (flat opening) and centrifuge the tubes at 195,000 g (SW40 rotor, 40,000 rpm in Beckman L-70 ultracentrifuge) for 2 h at 4° C. Following centrifugation, gently collect the plasma membrane layer fraction in the upper middle portion of the tube. Dilute the fraction with about 20 ml suspension buffer, and centrifuge at 100,000 g (35,000 rpm with 50.2 Ti rotor at Beckman L-70 ultracentrifuge) for 45 minutes at 4° C. Resuspend the resultant pellet in 1.0-1.5 ml of Storage buffer (sucrose 250 mM, Tris-HCl 10 mM, pH 7.4, containing leupeptin 10 μg/ml, aprotinin 10 μg/ml, and pepstatin A 2 μg/ml) and pass through a 25-gauge needle, 25 times, on ice. Aliquot 50 μg of the membrane preparations into Eppendorf tube and store at −80° C. for future use. Measure the protein concentration of obtained plasma membrane using Bio-Rad DC protein assay kit (microplate format).

### Example 3

### Experimental Procedure

One embodiment of the methods disclosed and claimed herein includes the following steps: a) add 89 μl of the solution of Tris-HCl buffer (10 mM, pH 7.4) and PMSF (400 μM) containing 8 μg membrane proteins (for batch V070302) to 96-well plate(s); b) add 10 μl of the bead suspension (10 mM Tris-HCl buffer, 0.1% NaN₃, 40 mg/ml beads) containing 400 μg WGA-Ysi beads; c) shake the plate(s) for 1 hour at room temperature; c) add 100 μl of test compound in 0.5% DMSO and 99.5% Tris-HCl (10 mM, pH 7.4) solution; d) add 1 μl of [³H]-vinblastine in 40% MeOH (0.04 μCi/μl); e) shake the plate for at least 1 hour at room temperature; and f) read in PerkinElmer MicroBeta counter 2 minutes/sample between 3 and 24 hours.

### Example 4

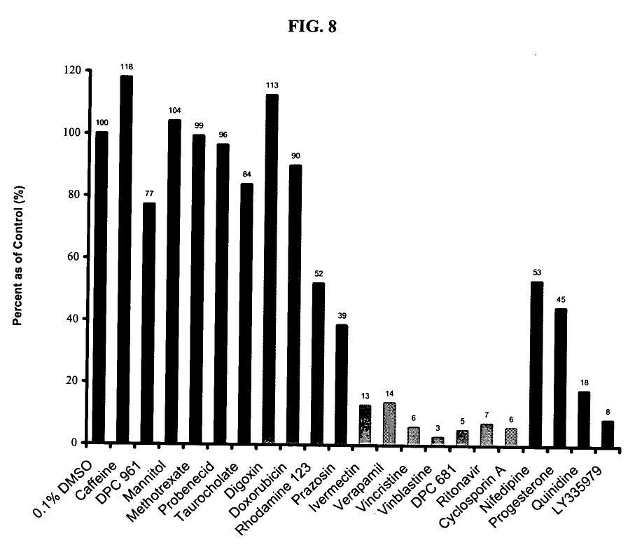

A total of 21 compounds including known P-gp substrates/inhibitors were tested. FIG. 8 shows the results from the selected compounds. Non-substrates, such as caffeine, have no impact on the P-gp signal as expected. Weak substrates, such as digoxin, do not appear to be able to alter the P-gp signal. However, a specific signal was strongly inhibited by compounds that are known as strong substrates/inhibitors, such as ivermectin/LY335979. It was observed that substrates that have higher affinities for other membrane transporters, such as methotrexate for MRP2, doxorubicin for MRP1, and probenecid/taurocholate for many organic anion transporters, did not appear to interfere with the P-gp signal, reflecting that the assay is specific for P-gp.

### Example 5

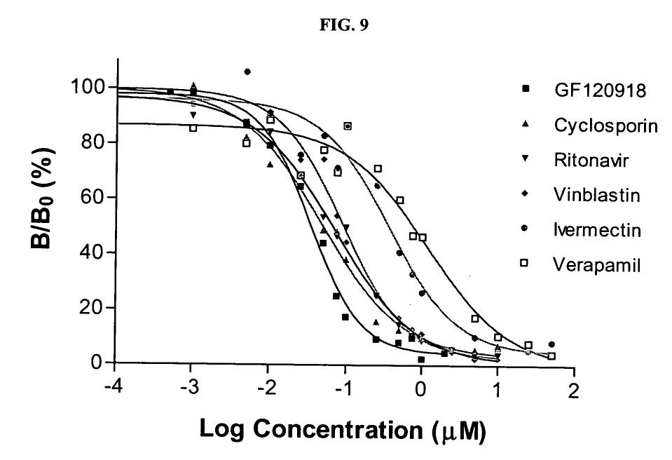

FIG. 9 shows the inhibitory profiles of selected compounds, and Table 1 lists corresponding IC₅₀ values. The rank order is in agreement with known potency data for P-gp inhibition by these compounds.

TABLE 1
IC₅₀ values determined by SPA for selected compounds

| Compound | IC₅₀ (nM) | R² |
|---|---|---|
| GF120918 | 34.3 | 0.9932 |
| Cyclosporine | 47.4 | 0.9879 |
| Ritonavir | 71.8 | 0.9925 |
| Vinblastine | 89.7 | 0.9909 |
| Ivermectin | 361.5 | 0.9705 |
| Verapamil | 1168.8 | 0.9559 |

### Example 6

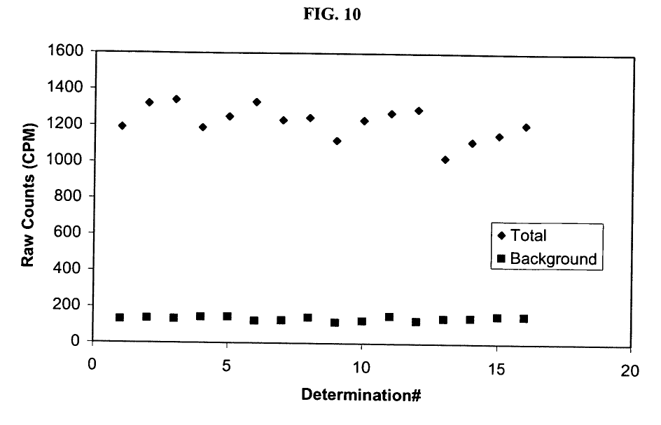

To evaluate the feasibility of the assay for high throughput screening for P-gp substrates/inhibitors, a limited validation study was performed. A total of 32 samples (16 radiolabeled-probes plus 100 μM non-labeled (cold) vinblastine as baseline control, and 16 radiolabeled probes alone to evaluate a specific signal) were prepared with KB-V1 membranes and beads following the procedure described in Example 3. The data obtained for the two sets of samples are shown in FIG. 10. Based on the values obtained in the experiment, several parameters were subjected to statistical analysis and are listed in Table 2. The statistical values reflect that the assay is amenable to a high throughput platform.

TABLE 2
List Of Statistical Parameters For High Throughput Platform

| | |
|---|---|
| Signal window | 12.8 |
| Z′ value | 0.73 |
| Signal/Noise | 12.3 |
| S/B | 8.9 |

While the subject matter disclosed and claimed herein has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

What is claimed is:

1. A method for identifying a test compound capable of modulating the activity of P-glycoprotein, comprising:
(a) incubating P-glycoprotein (P-gp) and scintillation proximity assay (SPA) beads under conditions which permit the formation of a complex between said P-gp and said SPA beads;
(b) introducing a test compound to said complex;
(c) introducing a radiolabeled probe to said complex; and
(d) measuring the energy produced by the interaction of said radiolabeled probe with said SPA beads to determine if said test compound is a modulator of P-gp.

2. The method of claim 1, further comprising the step of measuring the energy produced by the interaction of said radiolabeled probe with said SPA beads prior to said introduction of a test compound.

3. The method of claim 2, wherein said test compound is identified as a modulator of P-gp when the amount of energy produced in the absence of said test compound is different than the amount of energy produced in the presence of said compound.

4. The method of claim 1, wherein said energy is produced by a scintillant coated onto said SPA beads.

5. The method of claim 1, wherein said energy is light.

6. The method of claim 1, wherein said energy is measured by fluorescent, luminescent, radioactive, or absorbance readout.

7. The method of claim 1, wherein said radiolabeled probe is [³H]-vinblastine, or an analog thereof.

8. The method of claim 7, wherein said energy is produced at a [³H]-vinblastine, or vinblastine analog, concentration of between about 10 nM and about 1000 nM.

9. The method of claim 8, wherein said energy is produced at a [³H]-vinblastine, or vinblastine analog, concentration of about 30 nM.

10. The method of claim 1, wherein said P-gp is prepared from a human cell line.

11. The method of claim 10, wherein said human cell line is KB-V1.

12. The method of claim 1, wherein said SPA beads are yttrium silicate-wheat germ agglutinin (Ysi-WGA) beads.

13. The method of claim 1, wherein said SPA beads are present in an amount of between about 50 μg to about 2000 μg.

14. The method of claim 13, wherein said SPA beads are present in an amount of about 400 μg.

15. The method of claim 1, wherein said complex is formed by incubating said P-gp and said SPA beads in the presence of about 400 μM of phenyl-methylsulphonyl fluoride in about 50 mM Tris-HCl buffer.

16. The method of claim 1, wherein said complex is formed at a temperature of between about 4° C. and about 45° C.

17. The method of claim 16, wherein said complex is formed at a temperature of about 20° C.

18. The method of claim 1, wherein said incubation occurs for between about 0.01 hours and about 6 hours.

19. The method of claim 18, wherein said incubation occurs for about 1 hour.

20. The method of claim 1, wherein said test compound is a substrate, inhibitor, and/or a modulator of P-gp.

21. A method for identifying a test compound capable of modulating the activity of P-glycoprotein, comprising:
(a) incubating P-glycoprotein (P-gp) and scintillation proximity assay (SPA) beads coated with a scintillant under conditions which permit the formation of a complex between said P-gp and said SPA beads;
(b) introducing a test compound to said complex;
(c) introducing [³H]-vinblastine or a vinblastine analog; and
(d) measuring the light produced by the interaction of said [³H]-vinblastine or vinblastine analog, with said SPA beads to determine if said test compound is a modulator of P-gp.

22. The method of claim 21, wherein said SPA beads are yttrium silicate-wheat germ agglutinin (Ysi-WGA) beads.

23. A method for identifying a test compound capable of modulating the activity of P-glycoprotein, comprising:
(a) incubating P-glycoprotein (P-gp) and scintillation proximity assay (SPA) beads coated with a scintillant under conditions which permit the formation of a complex between said P-gp and said SPA beads, wherein said SPA beads are yttrium silicate-wheat germ agglutinin (Ysi-WGA) beads;
(b) introducing a test compound to said complex;
(c) introducing [³H]-vinblastine or a vinblastine analog; and
(d) measuring the light produced by the interaction of said [³H]-vinblastine or vinblastine analog, with said SPA beads to determine if said test compound is a modulator of P-gp.

24. A kit for performing the method of claim 1.

25. A kit for identifying a test compound capable of modulating the activity of P-glycoprotein, comprising:
(a) P-glycoprotein;
(b) scintillation proximity assay beads, and
(c) radiolabeled probe.

26. A kit of claim 25, wherein said radiolabeled probe is [³H]-vinblastine or a vinblastine analog.

27. A kit of claim 25, wherein said SPA beads are yttrium silicate-wheat germ agglutinin (Ysi-WGA) beads.

28. A kit for identifying a test compound capable of modulating the activity of P-glycoprotein, comprising:
(a) P-glycoprotein;
(b) yttrium silicate-wheat germ agglutinin beads, and
(c) [³H]-vinblastine or a vinblastine analog.
